(19) United States
(12) Patent Application Publication
(10) Pub. No.: US 2018/0146842 A1
(43) Pub. Date: May 31, 2018

(54) BOLUS REMOVAL DEVICE

(71) Applicant: UNITED STATES ENDOSCOPY GROUP, INC., Mentor, OH (US)

(72) Inventors: RONALD ALEXANDER SAHATJIAN, Lexington, MA (US); NATHANIEL M. BEALE, Hudson, MA (US); CHRISTOPHER J. KAYE, Concord, OH (US)

(73) Assignee: UNITED STATES ENDOSCOPY GROUP, INC., Mentor, OH (US)

(21) Appl. No.: 15/824,598

(22) Filed: Nov. 28, 2017

Related U.S. Application Data
(60) Provisional application No. 62/497,618, filed on Nov. 28, 2016.

(51) Int. Cl. A61B 1/00; A61B 1/015; A61B 1/273

(52) U.S. Cl. CPC A61B 1/00071; A61B 1/015; A61B 17/22; A61B 1/273; A61B 1/00128

(57) ABSTRACT

An endoscopic device includes: a hollow tube with a proximal end and a distal end. The proximal end and the distal end form a fluid communication therebetween. The proximal end is removably coupled with a distal tip of an endoscope and where coupled, forms a substantially airtight connection with the distal tip of the endoscope. The distal end removably contacts an object to be retrieved so as to deliver a suction force provided by the endoscope to facilitate removal of the object.

### CROSS-REFERENCE TO RELATED APPLICATIONS

This application claims priority to and any benefit of U.S. Provisional Patent Application No. 62/497,618, filed Nov. 28, 2016, the content of which is incorporated herein by reference in its entirety.

### TECHNICAL FIELD

The present subject matter relates generally to endoscopic treatment of obstructions in the esophagus. Specifically, the present subject matter pertains to endoscopic extraction of a food bolus, as a whole or piece-wise, from the esophagus using the suction provided by the endoscope.

### BACKGROUND

A food bolus is formed as part of the digestion process when chowed food is lubricated with saliva and formed into a cohesive mess. Usually, the bolus is swallowed and passed form the mouth through the esophagus into the stomach for gastric digestion. Sometimes, however, the bolus is lodged in the esophagus and does not pass to the stomach. In this instance, an esophageal food bolus obstruction is caused by the obstruction of the esophagus by an ingested foreign body. While some esophageal food boli can pass by themselves or with the assistance of medications, some require the use of endoscopy to push the obstructing food into the stomach, or remove it from the esophagus. A variety of devices are currently used to aid in the removal of esophageal foreign objects. There are needs to develop a new bolus removal device to overcome the shortcomings in prior art.

### SUMMARY

The present subject matter describes an endoscopic device, comprising: a hollow tube with a proximal end and a distal end. The proximal end and the distal end form a fluid communication therebetween. The proximal end is removably coupled with a distal tip of an endoscope and where coupled, forms a substantially airtight connection with the distal tip of the endoscope. The distal end removably contacts an object to be retrieved so as to deliver a suction force provided by the endoscope to facilitate removal of the object.

### BRIEF DESCRIPTION OF THE DRAWINGS

Features and advantages of the general inventive concepts will become apparent from the following detailed description made with reference to the accompanying drawings.

FIGS. 1a-1f are various views of an embodiment of the endoscopic device of the present subject matter;

FIGS. 2a-2f are various views of another embodiment of the endoscopic device;

FIGS. 3a-3e are various views of the third embodiment of the endoscopic device;

FIGS. 4a-4f are various views of the fourth embodiment of the endoscopic device; and

FIGS. 5a-5f are various views of the fifth embodiment of the endoscopic device.

### DETAILED DESCRIPTION OF INVENTION

This Detailed Description merely describes exemplary embodiments in accordance with the general inventive concepts and is not intended to limit the scope of the invention or the claims in any way. Indeed, the invention as described by the claims is broader than and unlimited by the exemplary embodiments set forth herein, and the terms used in the claims have their full ordinary meaning.

The general inventive concepts will now be described with occasional reference to the exemplary embodiments of the invention. This general inventive concept may, however, be embodied in different forms and should not be construed as limited to the embodiments set forth herein. Rather, these embodiments are provided so that this disclosure will be thorough and complete, and will fully convey the scope of the general inventive concepts to those skilled in the art.

Unless otherwise defined, all technical and scientific terms used herein have the same meaning as commonly understood by one of ordinary skill in the art encompassing the general inventive concepts. The terminology set forth in this detailed description is for describing particular embodiments only and is not intended to be limiting of the general inventive concepts. As used in this detailed description and the appended claims, the singular forms “a,” “an,” and “the” are intended to include the plural forms as well, unless the context clearly indicates otherwise.

Unless otherwise indicated, all numbers, such as for example, numbers expressing measurements or physical characteristics, used in the specification and claims are to be understood as being modified in all instances by the term “about.” Accordingly, unless otherwise indicated, the numerical properties set forth in the specification and claims are approximations that may vary depending on the suitable properties sought to be obtained in embodiments of the invention. Notwithstanding that the numerical ranges and parameters setting forth the broad scope of the general inventive concepts are approximations, the numerical values set forth in the specific examples are reported as precisely as possible. Any numerical values, however, inherently contain certain errors necessarily resulting from error found in their respective measurements.

As described above, a food bolus can obstruct the esophagus requiring the bolus to be pushed into the stomach, removed from the esophagus, or a combination of both. It is not always possible to pass an endoscope around a food bolus to confirm that it can be safely pushed into the stomach. In some patients, such as those with esophageal varices, strictures, esophageal malformations, tumors, eosinophilic esophagitis or other pathology, it may not be medically prudent to clear the bolus using the “push technique”. Alternatively, a snare or net device like the Roth Net® from United States Endoscopy Group Inc. may be used to remove the bolus from the esophagus. However, such a device must be positioned with its distal tip beyond the bolus before the net can be expanded and withdrawn. This may not be possible in a firmly impacted bolus situation. Attempts to pass the device beyond the bolus could also damage the esophageal mucosa, or could inadvertently push the bolus even deeper. Furthermore, piecemeal bolus removal involving multiple endoscope insertions can be lengthy procedures. Moreover, attempts to break the bolus into smaller portions using endoscopic accessories, such as graspers and snares, can be inefficient and labor intensive. Also, these devices face difficulties in breaking up an impacted bolus. In view of the above, there is a need to provide a device that can be used to quickly remove a bolus without the need to first break the impaction into smaller pieces.

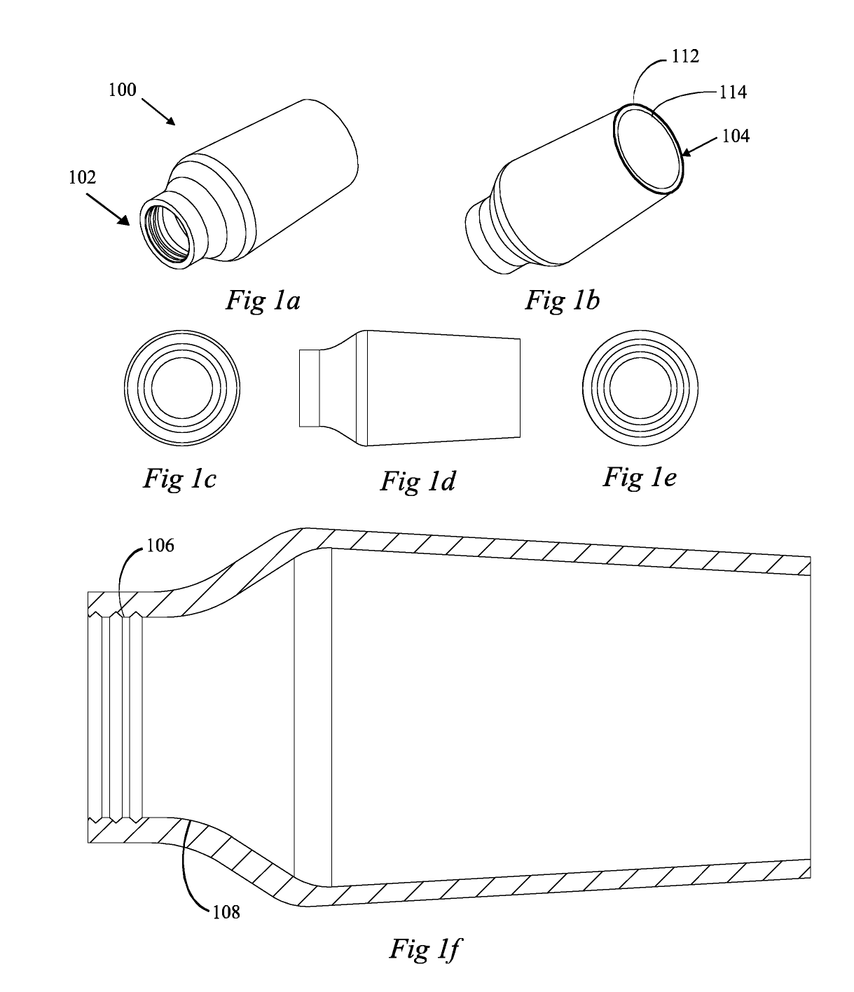

One aspect of the present subject matter discloses an endoscopic device 100 for eliminating an obstructed food bolus. Referring to FIGS. 1a-1f, the endoscopic device comprises a hollow tube with a proximal end 102 and a distal end 104. The hollow tube forms a fluid communication between the proximal end 102 and the distal end 104. The proximal end 102 is configured to couple with a distal tip of an endoscope and form an airtight connection. In some embodiments, the inner diameter of the proximal end 102 of the device 100 is sized to create a compression fit with the outer diameter of the distal tip of the endoscope. In some embodiments, the proximal end 102 comprises at least a groove 106. In some embodiments, the groove 106 is disposed at the interior surface 108 of the proximal end 102 of the endoscopic device 100. The groove is configured to help the device grip the distal tip of the endoscope.

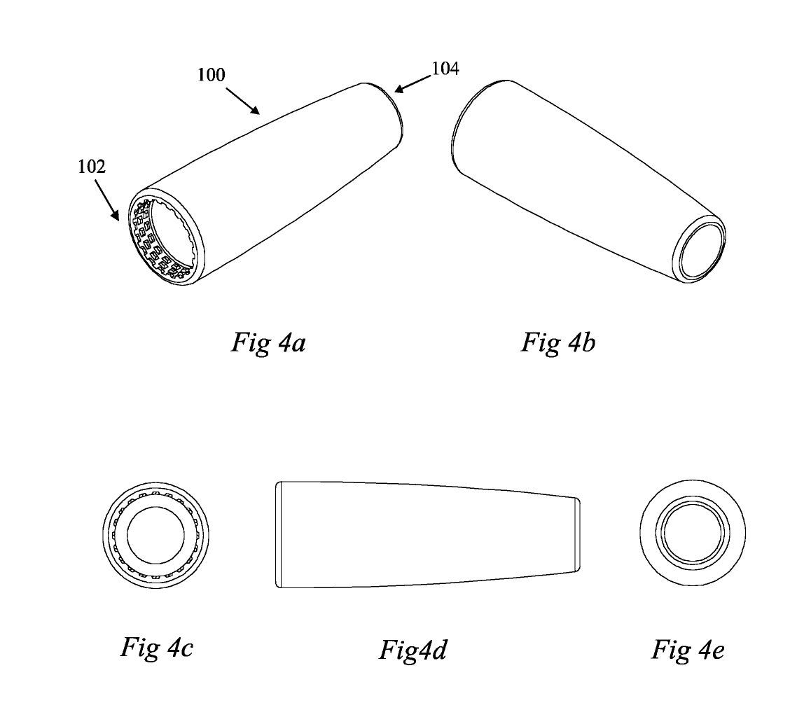

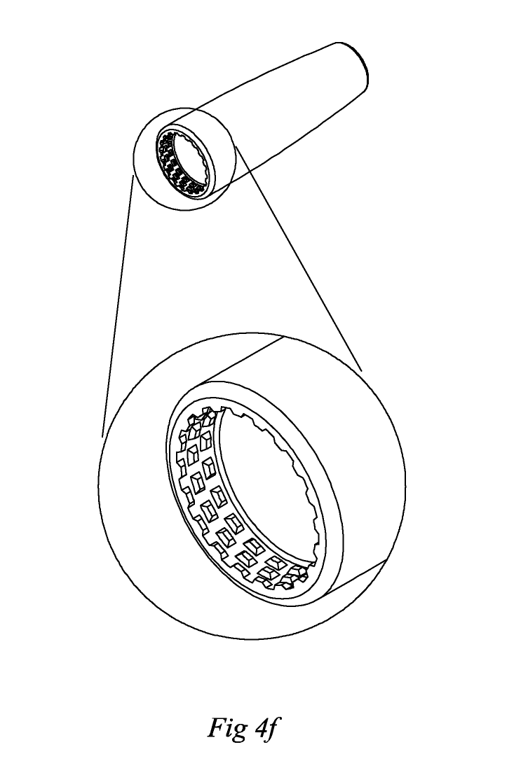

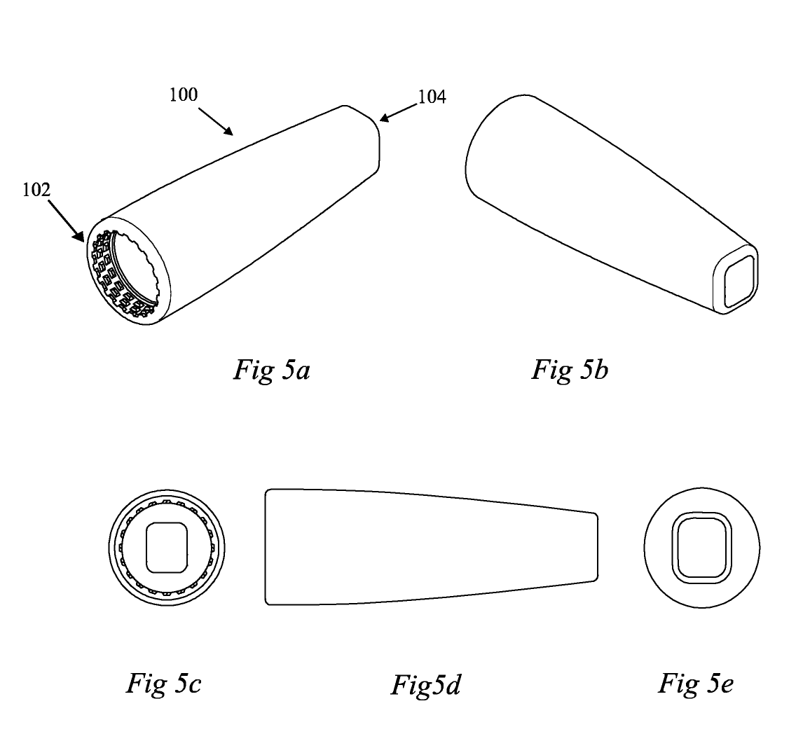

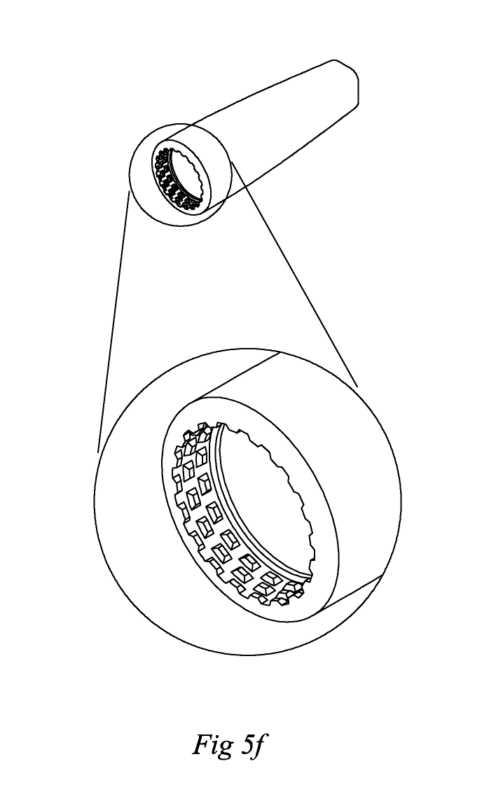

A person skilled in the art should reasonably understand that the proximal end of the device could be a number of known structures that ensure the device affixes or connects to the distal tip of the endoscope without causing any damages to the endoscope. For example, a series of dimples, divots, ribs, grooves, textured surfaces (shown in FIGS. 4f and 5f) or extremely smooth surfaces as well as external aids (such as tape) could be utilized to adequately anchor the device 100 to the distal tip of the endoscope and still preserve the air-tight requirement.

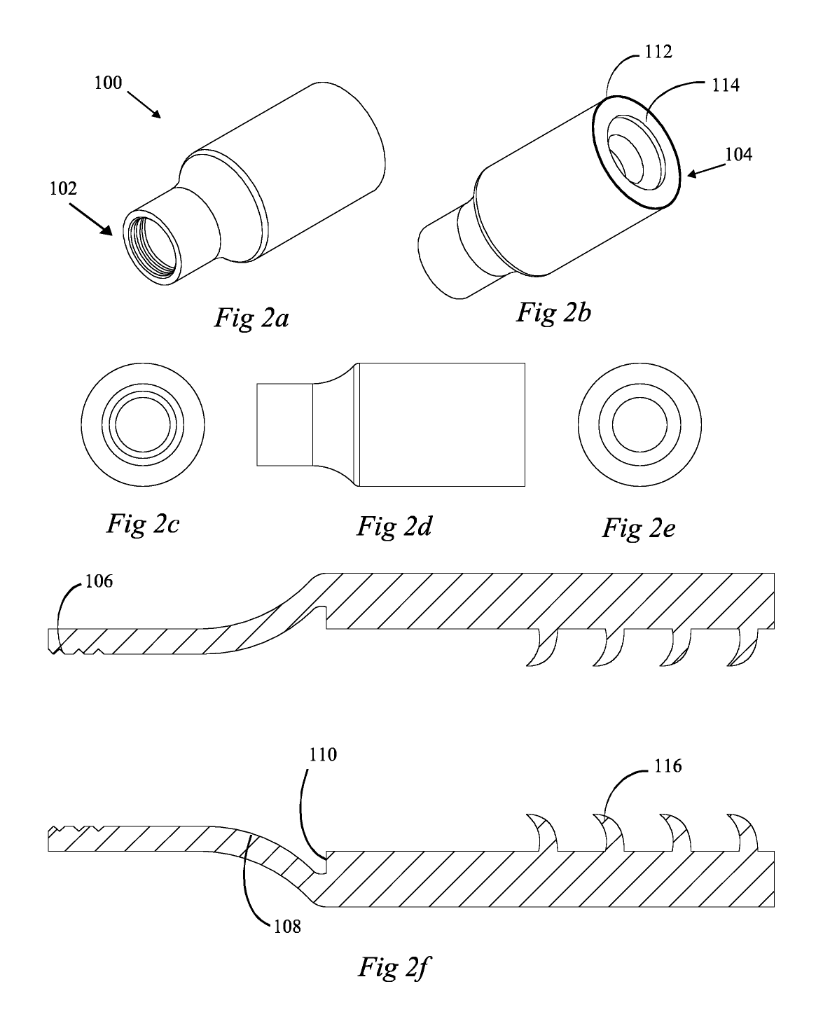

In some embodiments as shown in FIGS. 2a-2f, the device 100 further comprises a shoulder or stop 110 on the interior surface 108. The stop 110 prevents the distal tip of the endoscope from entering the device 100 too far. In some embodiments, the stop 110 helps a user to know when the device 100 has been pushed far enough onto the distal tip of the endoscope, so that a firm air tight connection has been made.

In some embodiments, the distal end 104 of the device 100 comprises a base perimeter 112. The base perimeter 112 of the distal end 104 is larger than the base perimeter of the distal end of the endoscope. In some embodiments, the distal end 104 of the device 100 comprises a base area 114. The base area 114 of the distal end 104 is larger than the base area of the distal end of the endoscope. The enlarged distal end 104 of the device 100 allows the user to grip a food bolus and remove the food bolus in the esophagus using the vacuum created via the suction lumen of the endoscope. The distal end 104 of the device 100 increases the surface area by which suction can be applied to the food bolus. In some other embodiments as shown in FIGS. 4a-4f and 5a-5f, the distal end of the device may be equal to or smaller than the distal tip of the endoscope.

The user attaches the endoscopic device 100 to the distal tip of the endoscope. The user positions the endoscope so that the distal end 104 of the device 100 is in contact with the surface of the food bolus. The user then activates the suction function at an endoscope so that the endoscope with the attached endoscopic device and the food bolus are then withdrawn as a single unit.

A person skilled in the art should understand that the endoscopic device 100 is able to grip similar or dissimilar objects from similar or dissimilar channels. It should also be noted that this device may prove beneficial for other medical procedures, such as the removal of blood pools or blood clots, or for procedures where a physical stand-off or increased device leverage is desired.

It should be noted that a food bolus or other similar foreign objects that can become lodged in the esophagus can consist of various materials, with a myriad of different textures, consistencies and structural properties. While the suction device is expected to provide adequate anchoring to the bolus, the user should take measures to provide airway protection via an over-tube, such as the Guardus® from U.S. Endoscopy. It should also be noted that the present subject matter could be combined with an over-tube, either as a stand-alone device or in a kit that contains an over-tube.

In some embodiments as shown in FIGS. 2a-2f, the interior surface of the device 100 comprises at least a tooth 116. The tooth 116 is a serrated tip, an alligator tooth, or a shark tooth. The tooth is to improve gripping the object and to hold the object in place until the object is removed from the body. In some embodiments, the device 100 comprises four teeth. A person skilled in the art should understand that the device could comprise other known structures to hold the object.

The endoscopic device 100 comprises a lateral wall having a thickness and strength sufficient to prevent collapse of the tubing when vacuum is applied via the endoscope. In some embodiments, the proximal end has the largest perimeter of the device. In some embodiments, the proximal end is not the largest perimeter of the device.

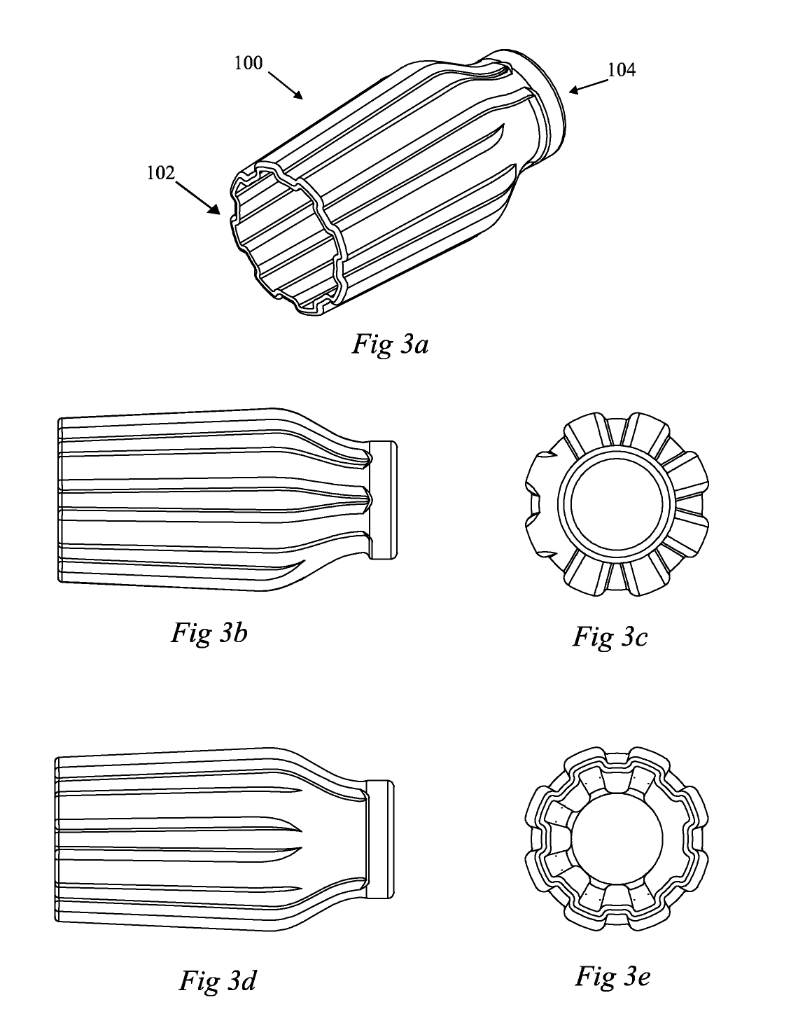

Referring to FIGS. 3a-3e, in some embodiments, the incorporation of support ribs and or indentations along either the internal and/or surface around a portion of the device (up to 320 degrees), would create a geometry that would preferentially flex to more efficiently contact a food bolus that is not necessarily in a plane perpendicular to the device. This could also allow differential pressure to be applied to the bolus (to compensate for variation in surface and/or density of the bolus) during contact and suction. One skilled in the art would also be able to create this effect with multiple zones (rather than one continuous one of 320 degrees) of varying stiffness around the device circumference.

It should be noted that the distal contact end of the device need not be round and that someone skilled in the art could design a shape that is obround, elliptical, etc. that could create adequate surface area for successful food bolus removal, as shown in FIGS. 5a-5f.

Another advantage of this device is that the endoscope accessory channel is still open, so that a device, such as a grasping tool (U.S. Endoscopy Mini-Raptor®, could be used to help position the bolus/scope for effective suction/anchoring.

The endoscopic device 100 has an atraumatic surface to avoid damages to esophagus. An intubation and/or an over-tube can also be used to protect the patient's airway during bolus removal.

In some embodiments, the endoscopic device 100 is transparent and visually clear to allow a user to see through the endoscopic device 100 at 360 degrees via an endoscope.

In some embodiments, the endoscopic device 100 is made of elastomeric polymer material, such as silicone, or any other elastomeric polymer material capable of gripping the endoscope with the proximal end and contacting the object with the distal end. For example, the device is made of Wacker LR 3000 Silicone with a durometer of 80 A. In some embodiments,

In some embodiments, the endoscopic device 100 is made in one-piece. In some embodiments, the endoscopic device 100 is made by more than one material. For example, the endoscopic device 100 is over-molded.

In some embodiments, the outer diameter of the endoscopic device is approximately 9 mm-15 mm. The inner diameter of the proximal end of the endoscopic device is appropriately sized to mate with a GI endoscope having a distal outer diameter of 8.6 mm-11.3 mm. In one embodiment, the length of the device is about 33.02 mm-33.53 mm. The diameter of the distal end is about 9.91 mm-10.41 mm. The inner diameter of the proximal end is about 7.87 mm-8.38 mm.

As noted above, the present invention relates generally to endoscopic treatment of obstructions in the esophagus and methods related thereto. The present invention should not be considered limited to the particular embodiments described above, but rather should be understood to cover all aspects of the invention as fairly set out in the appended claims. Various modifications, equivalent processes, as well as numerous structures to which the present invention may be applicable will be readily apparent to those skilled in the art to which the present invention is directed upon review of the present specification. The claims are intended to cover such modifications.

While various inventive aspects, concepts and features of the general inventive concepts are described and illustrated herein in the context of various exemplary embodiments, these various aspects, concepts and features may be used in many alternative embodiments, either individually or in various combinations and sub-combinations thereof. Unless expressly excluded herein all such combinations and sub-combinations are intended to be within the scope of the general inventive concepts. Still further, while various alternative embodiments as to the various aspects, concepts and features of the inventions (such as alternative materials, structures, configurations, methods, circuits, devices and components, alternatives as to form, fit and function, and so on) may be described herein, such descriptions are not intended to be a complete or exhaustive list of available alternative embodiments, whether presently known or later developed. Those skilled in the art may readily adopt one or more of the inventive aspects, concepts or features into additional embodiments and uses within the scope of the general inventive concepts even if such embodiments are not expressly disclosed herein. Additionally, even though some features, concepts or aspects of the inventions may be described herein as being a preferred arrangement or method, such description is not intended to suggest that such feature is required or necessary unless expressly so stated.

Still further, exemplary or representative values and ranges may be included to assist in understanding the present disclosure; however, such values and ranges are not to be construed in a limiting sense and are intended to be critical values or ranges only if so expressly stated. Moreover, while various aspects, features and concepts may be expressly identified herein as being inventive or forming part of an invention, such identification is not intended to be exclusive, but rather there may be inventive aspects, concepts and features that are fully described herein without being expressly identified as such or as part of a specific invention. Descriptions of exemplary methods or processes are not limited to inclusion of all steps as being required in all cases, nor is the order that the steps are presented to be construed as required or necessary unless expressly so stated.

1. An endoscopic device, comprising: a hollow tube with a proximal end and a distal end,
wherein the proximal end and the distal end form a fluid communication therebetween,
wherein the proximal end is removably coupled with a distal tip of an endoscope and where coupled, forms a substantially airtight connection with the distal tip of the endoscope, and
wherein the distal end removably contacts an object to be retrieved so as to deliver a suction force provided by the endoscope to facilitate removal of the object.

2. The endoscopic device of claim 1 further comprises a groove disposed at an interior surface of the proximal end.

3. The endoscopic device of claim 2 further comprises a stop, disposed between the groove and the distal end.

4. The endoscopic device of claim 1 further comprises dimples, divots, or texture at an interior surface of the proximal end.

5. The endoscopic device of claim 4 further comprises a stop disposed between the dimples, divots, or texture and the distal end.

6. The endoscopic device of claim 1 further comprises at least one tooth at an interior surface of the hollow tube.

7. The endoscopic device of claim 6, wherein the tooth is a serrated tip.

8. The endoscopic device of claim 1, wherein the endoscopic device is transparent.

9. The endoscopic device of claim 1, wherein the endoscopic device is made of elastomeric polymer material.

10. The endoscopic device of claim 1, wherein the endoscopic device is made in one-piece.

11. The endoscopic device of claim 1, wherein the distal end comprises a base perimeter larger than a base perimeter of the distal tip of the endoscope.

12. The endoscopic device of claim 1, wherein the distal end comprises a base perimeter smaller than a base perimeter of the distal tip of the endoscope.

13. The endoscopic device of claim 1, wherein the distal end comprises a base perimeter equal to a base perimeter of the distal tip of the endoscope.

14. The endoscopic device of claim 1, wherein the distal end comprises a base area larger than a base area of the distal tip of the endoscope.

15. The endoscopic device of claim 1, wherein the distal end comprises a base area smaller than a base area of the distal tip of the endoscope.

16. The endoscopic device of claim 1, wherein the distal end comprises a base area equal to a base area of the distal tip of the endoscope.

17. The endoscopic device of claim 1 further comprising elastomeric polymer material and non-elastomeric polymer material.

18. The endoscopic device of claim 1, wherein the endoscopic device is configured and sized to fit in an over-tube for the endoscopic device's safe removal without jeopardizing a patient's airway.
